(19) United States
(12) Patent Application Publication
(10) Pub. No.: US 2005/0154066 A1
(43) Pub. Date: Jul. 14, 2005

(54) Antiaging composition

(75) Inventors: Kenji Fujii, Kobe-shi (JP); Taizo Kawabe, Himeji-shi (JP); Hiroshi Kubo, Kobe-shi (JP); Keiichi Higuchi, Matsumoto-shi (JP)

Correspondence Address: SUGHRUE MION, PLLC, 2100 PENNSYLVANIA AVENUE, N.W., SUITE 800, WASHINGTON, DC, 20037, US

(73) Assignee: KANEKA CORPORATION

(21) Appl. No.: 11/029,493

(22) Filed: Jan. 6, 2005

Related U.S. Application Data
(60) Provisional application No. 60/559,966, filed on Apr. 7, 2004.

(30) Foreign Application Priority Data
Jan. 8, 2004 (JP) 2004-002863

(51) Int. Cl. A61K 31/12; A61K 31/075

(52) U.S. Cl. 514/690; 514/718

(57) ABSTRACT

The object of the present invention is to provide a composition which is effective in retarding or preventing the development of energy decrease, appearance change, etc. of humans and animals due to aging, and is highly safe even with a long period of taking.

The present invention relates to an antiaging composition which comprises reduced coenzyme Q as an active ingredient. By feeding mice which develop the aging symptom early (aging-accelerated model mice) with a feed containing reduced coenzyme Q₁₀ for a long period of time, aging process was prevented and retarded. Furthermore, aging-accelerated model mice fed with reduced coenzyme Q₁₀ for a long period of time showed no toxic symptom, thus it was found that the antiaging composition comprising a composition containing said substance can be made into a safe antiaging composition capable of being taken for a long period of time.

### TECHNICAL FIELD

The present invention relates to a composition capable of inhibiting or retarding aging.

### BACKGROUND ART

The antiaging effect has so far been considered from two aspects. One is the effect on apparent aging, namely skin aging and, specifically, this includes an improvement of wrinkles and freckles. This effect may include retention of skin elasticity and moisture, and the like effects. Mainly, preparations for external use such as cosmetics have claimed such effects, and some products have actually proved to be effective in humans. The other is the effect on bodily aging except for skin aging. As products effective in preventing bodily aging, antioxidant activity-based supplements may be mentioned. However, when examined in detail, the antiaging effects claimed by these supplements are just analogized from the effects obtained in vitro; and not demonstrated from the effects obtained in vivo. Among the in vitro effects shown by these supplements, for example, there are the lipid peroxidation inhibiting effect, the inhibitory effect on the formation of homocysteine whose content in the body may possibly increase with aging, and the like. However, any positive correlation between these effects in in vitro testing and the in vivo efficacy has not been confirmed. As regards the above-mentioned cosmetics for skin aging, etc., that the influence of oxidative stress on skin aging is great is credible to some extent as demonstrated by the fact of ultraviolet irradiation causing skin deterioration. As for the aging of the living body itself, however, it is considered that the analogy from a mere antioxidant activity is quite insufficient. Although a skin aging inhibitor containing ubiquinone (oxidized coenzyme Q) has been disclosed (Japanese Kohyo Publication 2002-513746), the document gives no description or suggestion about reduced coenzyme Q. Further, a method for the treatment of memory disorder resulting from aging which comprises administering carnitine, if necessary together with a coenzyme Q and/or creatine, is also known in the art (Japanese Kohyo Publication 2003-513039). In this method, however, carnitine is an essential ingredient. This publication does not mention at all about reduced coenzyme Q₁₀ or antiaging; in addition, it does not contain any specific report about an anti-mental disorder effect.

Oxidized coenzyme Q₁₀ is on sale as an antiaging supplement. In fact, however, its efficacy has not been demonstrated but is merely an analogy from the antioxidant activity of coenzyme Q₁₀. Further, reduced coenzyme Q₁₀ has not yet been commercialized because of its ready oxidizability; there is no knowledge in the art about its actual antiaging effect, like oxidized coenzyme Q₁₀.

### BRIEF DESCRIPTION OF THE DRAWINGS

FIG. 1 is a graph showing the increases in aging degree score in aging-accelerated model mice fed with reduced coenzyme Q₁₀. The ordinate denotes the aging degree score. The data were given in terms of mean±SD (n=10). The significant difference testing was performed in the manner of Student's t test. The mark * indicates that there is a significant difference relative to the control group at the degree of risk of 5%, and ** indicates that there is a significant difference relative to the control group at the degree of risk of 1%. The abscissa denotes the age of mice in months.

FIG. 2 is a graph showing the increases in aging degree score in aging-accelerated model mice fed with oxidized coenzyme Q₁₀. The ordinate denotes the aging degree score. The data were given in terms of mean±SD (n=10). The significant difference testing was performed in the manner of Student's t test. The mark * indicates that there is a significant difference relative to the control group at the degree of risk of 5%. The abscissa denotes the age of mice in months.

### DETAILED DESCRIPTION OF THE INVENTION

It is an object of the present invention to provide a substance or composition capable of preventing (retarding) aging.

The present inventors have made intensive investigations to solve the above-mentioned subjects, and as a result, they have found for the first time that reduced coenzyme Q₁₀ has a distinct aging-preventing (retarding) effect in the invention.

Thus, the present invention relates to
an antiaging composition
which comprises, as an active ingredient, a reduced coenzyme Q represented by the following formula (1): 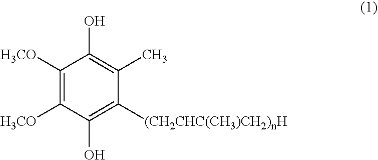
   in the formula, n represents an integer of 1 to 12.

Coenzyme Q is an essential component widely distributed in living organisms, from bacteria to mammals, and is known to occur as a component of the electron transport system of mitochondria within the cells of living bodies. Coenzyme Q functions as an electron carrier in the electron transport system through repeating the cycle of oxidation-reduction in mitochondria. In addition, it is known that reduced coenzyme Q shows antioxidant activity. In humans, coenzyme Q₁₀ in which the side chain of coenzyme Q has 10 repeating units is the main component. As mentioned above, reduced coenzyme Q₁₀ shows antioxidant activity in vitro but oxidized coenzyme Q₁₀ does not show antioxidant activity. However, it is believed that oxidized coenzyme Q₁₀ is converted to the reduced form by means of reductase in vivo.

As an important feature of coenzyme Q₁₀, its high safety may be mentioned. It is reported that, in a chronic toxicity test in rats, there was no toxic effect even when coenzyme Q₁₀ was administered every day for 52 weeks at 1,200 mg/kg/day (K. D. Williams et al., J. Agric. Food Chem. 47, 3756-3763, 1999). The dose of 1,200 mg/kg/day, when converted to the human basis (body weight 50 kg), corresponds to 60 g/day. Since the usual dose of coenzyme Q₁₀ used as a health food in Europe and America is 100 to 300 mg/day, it is evident that coenzyme Q₁₀ is a very highly safe supplement material.

Coenzyme Q comprises a reduced coenzyme Q represented by the following formula (1): 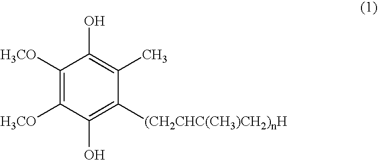
in the formula, n represents an integer of 1 to 12, and/or an oxidized coenzyme Q represented by the following formula (2): 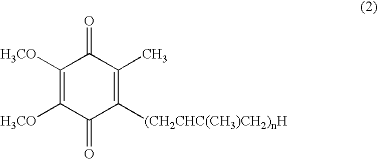
in the formula, n represents an integer of 1 to 12.

The method of obtaining oxidized coenzyme Q and reduced coenzyme Q is not particularly restricted. Employable are, for example, the method comprising obtaining coenzyme Q in the conventional manner such as synthesis, fermentation or extraction from a natural source, and then concentrating oxidized coenzyme Q fraction or reduced coenzyme Q fraction in the effluent by chromatography. When oxidized coenzyme Q is desired, methods known in the art can be used. When reduced coenzyme Q is desired, an ordinary reducing agent such as sodium borohydride or sodium dithionite (sodium hydrosulfite), is added to the above coenzyme Q according to need, and the coenzyme Q is reduced in the conventional manner to give reduced coenzyme Q, which is then concentrated by chromatography. Reduced coenzyme Q can also be obtained by the method comprising reacting an existing highly pure coenzyme Q product with such a reducing agent as mentioned above (the method described, for example, in Carpino, L. A. et al, 1989, J. Org. Chem. 54, 3303-3310).

The antiaging composition of the invention is a composition comprising reduced coenzyme Q as an active ingredient.

So long as the antiaging composition of the invention contains reduced coenzyme Q as an active ingredient, coenzyme Q is not particularly restricted but may consist of a reduced form alone or may be a mixture with a oxidized form.

Coenzyme Q that can be used in the practice of the invention may be any of those having the repeating units in the side chain (n in the formulas) of 1 to 12, as illustrated by the above formulas (1) and (2). Among them, however, one having the repeating units in the side chain of 10 (n in the above formulas (1) and (2) being 10), namely coenzyme Q₁₀, is particularly preferably used.

In cases where coenzyme Q is a mixture of the reduced and oxidized forms, the content of reduced coenzyme Q is preferably not lower than 20% by weight, more preferably not lower than 40% by weight, still more preferably not lower than 50% by weight, relative to the total amount of coenzyme Q. Generally, the upper limit is preferably not higher than 99.5% by weight, but may be not higher than 95% by weight.

In the antiaging composition of the invention, the content of reduced coenzyme Q is preferably 0.001 to 99% by weight, more preferably 0.01 to 99% by weight, still more preferably 0.1 to 50% by weight, relative to the whole composition.

The antiaging composition of the invention may contain, in addition to coenzyme Q, various additives acceptable from the medical or food hygiene law or the like viewpoint. In cases where it is used as a measure for various diseases, medicaments for the disease(s) concerned can be used in combination.

The above-mentioned additives are not particularly restricted but include, for example, excipients/diluents, disintegrants, lubricants, binders, coating agents, colorants, coagulation inhibitors, absorption promoters, solubilizing agents, stabilizers, health food materials, nutritional supplement materials (supplement materials), and the like.

The excipients/diluents are not particularly restricted but include, for example, white sugar, lactose, glucose, corn starch, mannitol, crystalline cellulose, calcium phosphate, calcium sulfate, and the like.

The disintegrants are not particularly restricted but include, for example, starch, agar, calcium ditrate, calcium carbonate, sodium hydrogen carbonate, dextrin, crystalline cellulose, carboxymethylcellulose, tragacanth, and the like.

The lubricants are not particularly restricted but include, for example, talc, magnesium stearate, polyethylene glycol, silica, hydrogenated vegetable oils, and the like.

The binders are not particularly restricted but include, for example, ethylcellulose, methylcellulose, hydroxypropymethyllcellulose, tragacanth, shellac, gelatin, gum arabic, polyvinylpyrrolidone, polyvinyl alcohol, polyacrylic acid, polymethacrylic acid, sorbitol, and the like.

The coating agents are not particularly restricted but include, for example, gum arabic, Opadry, prunella spike, castor wax, carboxyvinyl polymers, carmellose, hydrous silicon dioxide, magnesium silicate, vinyl acetate resins, stearic acid, cetanol, hydroxypropylmethylcellulose, and the like.

The colorants are not particularly restricted but those authorized to be added to drugs and/or foods, and the like can be used, for example.

The coagulation inhibitors are not particularly restricted but include, for example, stearic acid, talc, light silicic anhydride, hydrous silicon dioxide, and the like.

The absorption promoters are not particularly restricted but include, for example, higher alcohols, higher fatty acids, surfactants such as glycerol fatty acid esters, and the like.

The solubilizing agents are not particularly restricted but include, for example, organic acids such as fumaric acid, succinic acid, malic acid, and the like.

The stabilizers are not particularly restricted but include, for example, benzoic acid, sodium benzoate, ethyl parahydroxybenoate, and the like.

The health food materials are not particularly restricted but include, for example, Kampo (Chinese) medicines (e.g. Irei-to (crude drug extract for treating stomach disorder, etc.), Unkei-to (crude drug extract for warming body, etc.), Unsei-in (crude drug extract for promoting blood circulation, etc.), Ogi-kenchu-to (extract of Astragalus membranaceus, etc.), Oren-gedoku-to (crude drug extract for reducing fever, inflammation, etc.), Oren-to (extract of Coptis chinensis, etc.), Kakkon-to (extract of Puerariae radix, etc.), Kami-kihi-to (crude drug extract for treating anemia, insomnia, neurosis, etc.), Kami-shoyo-san (crude drug extract for treating menstrual and climacteric disorder, etc.), Kam-baku-taiso-to (crude drug extract for treating night cry, convulsion, etc.), Kikyo-to (extract of Platycodon grandiflorum, etc.), Kihi-to (crude drug extract for treating anemia, insomnia, etc.), Kumi-binro-to (extract of nine crude drug species, e.g. Livistona chinesis var. subglobosa), Keigai-rengyo-to (crude drug extract for treating chronic paranasal sinusitis, acne, etc.), Keishi-ka-shakuyaku-daio-to (crude drug extract for treating stomach disorder, etc.), Keishi-ka-shakuyaku-to (extract of Cinnamomi cortex, Chinese peony, etc.), Keishi-ka-ryukotsu-borei-to (extract of Cinnamomi cortex, Ostrea gigas, etc.), Keishi-to (extract of Cinnamomi cortex, etc.), Keishi-ninjin-to (extract of Cinnamomi cortex, gensing, etc.), Keishi-bukuryo-gan (extract of Cinnamomi cortex, Poria cocos, etc.), Keihi-to (crude drug extract for treating digestive trouble, diarrhea, etc.), Koso-san (extract of Cyperus rotundus, etc.), Goko-to (crude drug extract for treating cough, asthma, etc.), Goshaku-san (crude drug extract for treating blood and water circulation, etc.), Gosha-jinki-gan (crude drug extract for improving body function, etc.), Gorin-san (crude drug extract for treating frequent urination, miction pain, etc.), Saikan-to (extract of Bupleurum chinense, etc.), Saiko-ka-ryukotsu-borei-to (extract of Bupleurum chinense, Ostrea gigas, etc.), Saiko-keishi-kankyo-to (extract of Bupleurum chinense, Cinnamomi cortex, Zingiber officinale, etc.), Saiko-keishi-to (extract of Bupleurum chinense, Cinnamomi cortex, etc.), Saiko-seikan-to (extract of Bupleurum chinense, Scutellaria baicalensis Georgi, etc.), Saiboku-to (extract of Bupleurum chinense, Scutellaria baicalensis Georgi, Pinellia ternate, etc.), Sairei-to (crude drug extract for reducing inflammation, improving water circulation, etc.), Sansonin-to (extract of Zizyphus jujuba, etc.), Jiin-koka-to (crude drug extract for tempering cough), Shigyaku-san (extract of Bupleurum chinense, etc.), Shikunshi-to (crude drug extract for improving stomach function, etc.), Shimotsu-to (extract of four crude drug species e.g. Angelica sinensis, etc.), Sha-kanzo-to (crude drug extract for tempering palpitation and breath shortness, etc.), Shakuyaku-kanzo-to (extract of Paeonia lactiflora and Glycyrrhiza uralensis), Juzen-taiho-to (crude drug extract for recovering vitality and energy), Jumi-haidoku-to (extract of ten crude drug species and used for dermatitis, etc.), Sho-kenchu-to (crude drug extract for improving stomach function, etc.), Sho-saiko-to (crude drug extract for protecting liver, etc.), Sho-seiryu-to (crude drug extract for treating allergic rhinitis, asthma, etc.), Shofu-san (crude drug extract for treating eczema), Shin'i-seihai-to (extract of Magnolia kobus, etc.), Shimpi-to (extract of Ephedra sinica, etc.), Shimbu-to (crude drug extract for warming body to improve body function, etc.), Seijo-bofu-to (crude drug extract for treating acne), Seisho-ekki-to (crude drug extract for treating summer weariness, etc.), Seishin-renshi-in (crude drug extract for treating urination disorder, etc.), Seihai-to (extract of Magnolia praecocissima, etc.), Sokei-kakketsu-to (crude drug extract for reducing nerve pain, backache, etc.), Daio-kanzo-to (extract of Rheum tanguticum and Glycyrrhiza uralensis), Daio-botampi-to (extract of Rheum tanguticum, Paeonia suffruticosa, etc.), Dai-kenchu-to (crude drug extract for reducing stomachache, etc.), Dai-saiko-to (extract of Bupleurum chinense, Scutellaria baicalensis Georgi, etc.), Dai-saiko-to-kyo-daio (extract of Bupleurum chinense, Scutellaria baicalensis Georgi, Pinellia ternate, etc.), Dai-joki-to (crude drug extract for treating constipation, etc.), Dai-bofu-to (extract of Ledebouriella seseloides, etc.), Ji-daboku-ippo (crude drug extract for treating bruise), Choi-joki-to (crude drug extract for loosening the bowels, etc.), Choto-san (extract of Uncaria rhynchophylla, etc.), Choyo-to (crude drug extract for reducing hypogastric region pain), Chorei-to (extract of Polyporus umbellatus, etc.), Chorei-to-go-shimotsu-to (extract of Polyporus umbellatus, etc.), Tsu-do-san (crude drug extract for treating menstrual disorder, menstrual cramps, etc.), Tokaku-joki-to (crude drug extract for treating constipation, menstrual disorder, etc.), Toki-inshi (crude drug extract for treating eczema, dry skin itch, etc.), Toki-kenchu-to (extract of Angelica sinensis, Cinnamomi Cortex, Paeonia lactiflora, etc.), Toki-shakuyaku-san (extract of Angelica sinensis, Paeonia lactiflora, etc.), Toki-to (extract of Angelica sinensis, etc.), Nichin-to (crude drug extract for reducing nausea, vomiting, etc.), Nyoshin-san (crude drug extract for treating flash, dizziness, etc.), Ninjin-to (extract of gensing, etc.), Ninjin-yoei-to (extract of gensing, Astragalus membranaceus, etc.), Haino-san-kyu-to (crude drug extract for treating skin tumor, etc.), Bakumondo-to (extract of Ophiopogon japonicus, etc.), Hachimi-jio-gan (extract of eight crude drug species e.g. Rehmannia glutinosa, etc.), Hange-koboku-to (extract of Pinellia ternate, Magnolia officinalis, etc.), Hange-shashin-to (extract of Pinellia ternate, etc.), Byakko-ka-ninjin-to (extract of Gypsum, etc.), Bukuryo-in (extract of Poria cocos, etc.), Bukuryo-in-go-hange-koboku-to (crude drug extract for reducing emesis of pregnancy, etc.), Heii-san (crude drug extract for treating heavy stomach, etc.), Boi-ogi-to (extract of Aristolochia fangchi, Astragalus membranaceus, etc.), Bofu-tsusho-san (extract of Ledebouriella seseloides, etc.), Hochu-ekki-to (crude drug extract for improving stomach function to invigorate, etc.), Mao-to (extract of Ephedra sinica, etc.), Mao-bushi-saishin-to (extract of Ephedra sinica, Aconitum carmichaeri, etc.), Ma-kyo-kan-seki-to (extract of Ephedra sinica, Prunus armeniaca, etc.), Mashinin-gan (extract of Cannabis sativa, etc.), Moku-boi-to (extract of Cocculus trilobus, etc.), Yoku-kan-san (crude drug extract for reducing nerve excitement, etc.), Yoku-kan-san-ka-chimpi-hange (crude drug extract for reducing nerve excitement, etc.), Rikkunshi-to (crude drug extract for reducing heavy stomach, etc.), Rikko-san (crude drug extract for reducing toothache, etc.), Ryutan-shakan-to (extract of Gentiana scabra, etc.), Ryo-kan-kyo-mi-shin-ge-nin-to (crude drug extract for controlling cough and clearing throat, etc.), Rokumi-gan (extract of six crude drug species and used for improving body function, etc.), and the like, tea leaves (e.g. green tea, Japanese tea mixed with roasted rice, powdered green tea, green tea of middle grade, toasted tea, roasted tea, jasmine tea, oolong tea, tea, black tea, flower tea, blue tea, white tea, etc.), herbs (e.g. Italian parsley, elecampane, olive, oregano, cardoon, chamomile, curry plant, catnip, caraway, Christmas rose, crimson clover, cornflower, common mallow, salad burnet, santolina, cinnamon, jasmine, stevia, sage, linden (lime), scented geranium, St.-John's-wort, soapwort, Solomon's seal, thyme, tansy, chervil, chive, nasturtium, nutmeg, basil, honeysuckle, hyssop, flax, fennel, foxglove, black hollyhock, French marigold, betony, heliotrope, bergamot, hemp agrimony, rue, pot marigold, borage, white horehound, myrtle, mullein, marjoram, mint, yarrow, lavender, Lady's bedstraw, lemongrass, lemon verbena, lemon balm, rose, rosemary, rocket, wild strawberry, wild pansy, forget-me-not, etc.), pycnogenol, flavangenol, propolis, gingko leaves, royal jelly, carnitine, mushrooms, chlorophyll juice, extracts from these, and the like.

The nutritional supplement materials are not particularly restricted but include, for example, amino acids, metal ions, proteins, saccharides, fatty acids, yeast extracts, vegetable extracts, fish meat extracts, fruits, fruit extracts, and the like.

The antiaging composition of the invention may contain another antioxidant and the like.

The antioxidant includes, but is not limited to, for example, citric acid, citric acid derivatives, vitamin C, vitamin C derivatives, lycopene, vitamin A, carotenoids, vitamin B, vitamin B derivatives, flavonoids, polyphenols, glutathione, selenium, sodium thiosulfate, vitamin E, vitamin E derivatives, pyrroloquinoline quinone, pyrroloquinoline quinone derivatives, superoxide dismutase (SOD), glutathione peroxidase, glutathione-S-transferase, glutathione reductase, catalase, ascorbic acid peroxidase, mixtures of these, and the like.

The method of producing the antiaging composition of the invention comprises mixing reduced coenzyme Q represented by the above formula (1) with acceptable additives.

Thus, the above-mentioned antiaging composition can be prepared, for example, by adding, mixing, spraying, including, conjugating, etc. the above-mentioned additives, antioxidants and the like, according to need, to/with reduced coenzyme Q obtained as mentioned above. Further, according to the desired dose form as mentioned below, the composition can also be obtained by adding various additives, solvents, bases and the like, which are accepted as pharmaceutical and/or food additives, to reduced coenzyme Q and subjecting the resulting mixture to processing for preparing various adequate dosage forms, namely to granulation, coating, microencapsulation, inclusion, incorporation, emulsification, suspension, or the like.

The dosage form of the antiaging composition of the invention may be either liquid or solid. As for the method of administration, various methods are employable such as oral administration, injection, nasal administration, administration in the form of an ophthalmic solution or suppositories, or eating of a coenzyme Q-containing foodstuff. Generally, oral administration is considered to be most effective method from the dosage and the like viewpoint. When oral administration is difficult, the composition of the invention can be administered by any other route than oral administration without any problem. For example, in patients or aged persons who find difficulty in orally taking nutrients, the recommendable method of administration includes, but is not limited to, administration in the form of suppositories, external preparations for dermal application, or the like.

In the case of oral administration, for example, the dose of the antiaging composition of the invention as expressed in terms of the amount of coenzyme Q is preferably 30 to 1,200 mg, more preferably 50 to 800 mg, still more preferably 100 to 300 mg, per day per human.

The antiaging composition of the invention can prevent or retard the aging of humans and/or animals.

The term “aging” as used herein refers, for example, to blunting of behavior, for example decreased activity or decreased passivity, backbone bending, loss of hair, corneal clouding, inflammation periphery of the eye and/or ear, and the like.

The administration of the antiaging composition of the invention can delay the onset of, or ameliorate, these aging-associated symptoms.

In evaluating such antiaging effects, for example, aging-accelerated model mice, can be used as an in vivo evaluation model. This mouse is a model mouse discovered and bred at Kyoto University, Japan, which develops the state of aging early and markedly. This mouse shows the state of aging quite similar to that of humans, and is a useful model animal for in vivo testing for antiaging effects.

The method of preventing aging in an animal according to the invention comprises administering the above antiaging composition to the target animal.

The animal includes mammals, fish, birds, reptiles, insects, and the like. Preferred as the animal are mammals, for example humans.

The term “administering” includes, within the meaning thereof, topical, enteral, e.g. oral or rectal, or parenteral administration. As for the route of administration, oral administration is preferred.

The reduced coenzyme Q-containing composition of the invention produces excellent effect on preventing or retarding aging.

### BEST MODE FOR CARRYING OUT THE INVENTION

The following examples illustrate the present invention in further detail. These examples are, however, by no means limitative of the scope of the invention.

The purity and reduced coenzyme Q₁₀/(reduced coenzyme Q₁₀+oxidized coenzyme Q₁₀) ratio (weight ratio) were determined by the following HPLC analysis.

(HPLC Analysis Conditions)

Column: SYMMETRY C18 (product of Waters Corporation), 250 mm (length), 4.6 mm (inner diameter); mobile phase: C₂H₅OH:CH₃OH=4:3 (v:v); detection wavelength: 210 nm; flow rate: 1 ml/min.; retention time for reduced coenzyme Q₁₀: 9.1 min., retention time for oxidized coenzyme Q₁₀: 13.3 min.

### PRODUCTION EXAMPLE 1

### Production of Reduced Coenzyme Q₁₀

100 g of oxidized coenzyme Q₁₀ (purity 99.4%) and 60 g of L-ascorbic acid were added to 1,000 g of ethanol, and the mixture was subjected to the reduction reaction while stirring at 78° C. After the lapse of 30 hours, the mixture was cooled to 50° C. and, while maintaining that temperature, 330 g of ethanol and 70 g of water were added. This ethanol solution (containing 100 g of reduced coenzyme Q₁₀) was cooled to 2° C. at a cooling rate of 10° C./hour with stirring to give a white slurry. The slurry obtained was filtered under reduced pressure, the wet crystals were washed in sequence with cold ethanol, cold water and cold ethanol (the temperature of the cold solvents used being 2° C.), and the wet crystals were further dried under reduced pressure (20 to 40° C., 1 to 30 mmHg) to give 97 g of reduced coenzyme Q₁₀ (containing about 1% of oxidized coenzyme Q₁₀) as white dry crystals. All the operations other than drying under reduced pressure were carried out in a nitrogen atmosphere.

### PRODUCTION EXAMPLE 2

### Production of Reduced Coenzyme Q₁₀

100 g of oxidized coenzyme Q₁₀ was dissolved in 1,000 g of heptane at 25° C. While stirring, the solution was gradually added with an aqueous solution prepared by dissolving 100 g of sodium dithionite (purity not lower than 75%) as a reducing agent in 1,000 ml of water, thus allowing the reduction reaction to proceed at pH 4 to 6 at 25° C. After the lapse of 2 hours, the aqueous phase was removed from the reaction mixture, and the heptane phase was washed with 1,000 g of a deaerated saturated aqueous solution of sodium chloride for six times. This heptane phase was subjected to solvent substitution under reduced pressure for preparing a 7% (w/w) ethanol solution of reduced coenzyme Q₁₀ at 50° C. (the solution containing 100 g of reduced coenzyme Q₁₀). 50 g of water was added to this ethanol solution, and the mixture was cooled to 2° C. at a cooling rate of 10° C./hour while stirring to precipitate crystals. All the operations were carried out in a nitrogen atmosphere. The slurry thus obtained was filtered under reduced pressure, and the wet crystals were washed in sequence with cold ethanol, cold water and cold ethanol (the temperature of the cold solvents used for washing being 2° C.). The wet crystals were further dried under reduced pressure (20 to 40° C., 1 to 30 mmHg) to give 97 g of reduced coenzyme Q₁₀ (containing about 1% of oxidized coenzyme Q₁₀) as white dry crystals.

### EXAMPLE 1

### Aging Preventing (Retarding) Effect in Aging-Accelerated Model Mice

Aging-accelerated model mice (SAMP1, 3-week-old females) were allowed free access to a feed (CE-2, product of CLEA Japan, Inc.) containing 0.2% reduced coenzyme Q₁₀ (containing about 1% of oxidized coenzyme Q₁₀) obtained in Production Example 1, and the aging degree of each mouse was quantified with time according to the aging degree score mentioned below. The dose of reduced coenzyme Q₁₀ as estimated from the feed consumption and animal weight corresponded to about 150 to 250 mg/kg/day. A control group was given the feed alone (CE-2, product of CLEA Japan, Inc.).

The aging-accelerated model mice used were model mice discovered and bred at Kyoto University which develop the state of aging early and markedly. These mice show the state of aging quite similar to that of humans, and are useful model animals for in vivo testing for antiaging effects. The aging degree scoring system employed was the scoring system established by the Council for SAM Research. Thus, scores of 0 to 4 were given to each animal with respect to each of the following 11 items: 1: decrease in activity (searching behavior), 2: decrease in passivity (pinching escaping behavior), 3: loss of hair luster, 4: roughening of hair, 5: loss of hair, 6: skin ulcer, 7: periophthalmic lesions (blepharitis and periophthalmic erosion), 8: corneal clouding, 9: corneal ulcer, 10: cataract, 11: increased anterior or backward spinal curvature. The conditions found in a group of young mice about 3 months of age were taken as standards and given the score 0 (zero). Thus, a higher score indicates more advanced aging.

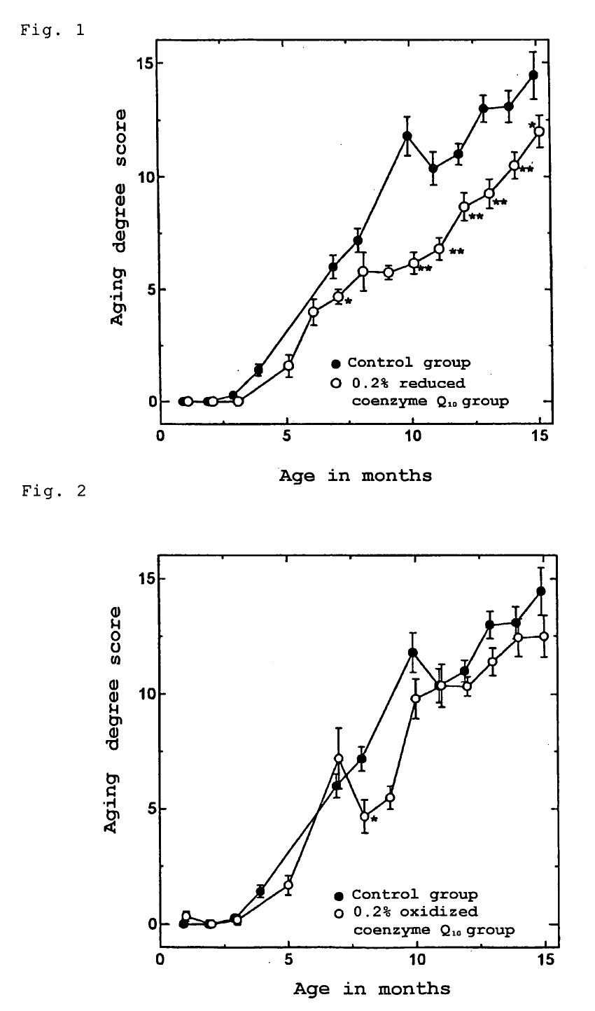

The results are shown in FIG. 1. As can be seen in FIG. 1, the aging degree scores could be distinctly prevented from increasing as a result of taking of reduced coenzyme Q₁₀.

### COMPARATIVE EXAMPLE 1

The test of Example 1 was performed in the same manner except that oxidized coenzyme Q₁₀ was used in lieu of reduced coenzyme Q₁₀. The dose of oxidized coenzyme Q₁₀ as estimated from the feed consumption and animal body weight corresponded to about 150 to 250 mg/kg/day, and it was nearly the same as the dose of reduced coenzyme Q₁₀. The results are shown in FIG. 2. As a result of taking of oxidized coenzyme Q₁₀, the increases in aging degree score were suppressed to a certain extent but the aging preventing effect thereof was weak as compared with that of reduced coenzyme Q₁₀.

### PREPARATION EXAMPLE 1

### Powder

Reduced coenzyme Q₁₀ (containing about 1% of oxidized coenzyme Q₁₀) was dissolved in propanol and allowed to be adsorbed on microcrystalline cellulose, which was then dried under reduced pressure. This was mixed with corn starch in a nitrogen flow to give a powder preparation.

| | |
|---|---|
| Reduced coenzyme Q₁₀ | 9.9 parts by weight |
| Oxidized coenzyme Q₁₀ | 0.1 parts by weight |
| Microcrystalline cellulose | 40 parts by weight |
| Corn starch | 55 parts by weight |

### PREPARATION EXAMPLE 2

### Capsules

Using the materials specified below, a powder preparation was prepared in the same manner as in Preparation Example 1. This powder was filled into gelatin capsules in the conventional manner. The filled capsules were sealed and packed in a nitrogen atmosphere and stored in a refrigerator.

| | | |
|---|---|---|
| Reduced coenzyme Q₁₀ | 19.8 | parts by weight |
| Oxidized coenzyme Q₁₀ | 0.2 | parts by weight |
| Microcrystalline cellulose | 40 | parts by weight |
| Corn starch | 20 | parts by weight |
| Lactose | 65 | parts by weight |
| Magnesium stearate | 3 | parts by weight |
| Polyvinylpyrrolidone | 2 | parts by weight |

### PREPARATION EXAMPLE 3

### Soft Capsules

Corn oil was warmed to 50° C. Thereto was added with reduced coenzyme Q₁₀ (containing about 1% of oxidized coenzyme Q₁₀) melted at the same temperature for dissolution. The solution was encapsulated into soft capsules in the conventional manner.

| | | |
|---|---|---|
| Reduced coenzyme Q₁₀ | 49.5 | parts by weight |
| Oxidized coenzyme Q₁₀ | 0.5 | parts by weight |
| Corn oil | 350 | parts by weight |

### PREPARATION EXAMPLE 4

### Tablets

Reduced coenzyme Q₁₀ (containing about 1% of oxidized coenzyme Q₁₀) was dissolved in propanol and allowed to be adsorbed on microcrystalline cellulose, which was then dried under reduced pressure. This was mixed with corn starch, lactose, carboxymethylcellulose calcium and magnesium stearate in a nitrogen atmosphere, an aqueous solution of polyvinylpyrrolidone was then added as a binder, to the resulting mixture, and the whole mixture was granulated in the conventional manner. To this granulation product, talc was added and mixed as a lubricant, and then the resulting mixture was made into tablets. The tablets were packed in a nitrogen atmosphere and stored in a refrigerator.

| | | |
|---|---|---|
| Reduced coenzyme Q₁₀ | 19.8 | parts by weight |
| Oxidized coenzyme Q₁₀ | 0.2 | parts by weight |
| Corn starch | 25 | parts by weight |
| Lactose | 15 | parts by weight |
| Carboxymethylcellulose calcium | 10 | parts by weight |
| Microcrystalline cellulose | 40 | parts by weight |
| Polyvinylpyrrolidone | 5 | parts by weight |
| Magnesium stearate | 3 | parts by weight |
| Talc | 10 | parts by weight |

The reduced coenzyme Q-containing composition of the invention produces excellent effect on preventing or retarding aging.

1. An antiaging composition
which comprises, as an active ingredient, a reduced coenzyme Q represented by the following formula (1): 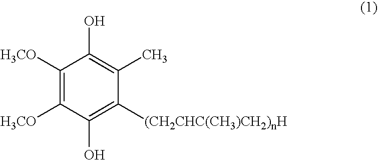
in the formula, n represents an integer of 1 to 12.

2. The antiaging composition according to claim 1,
wherein the reduced coenzyme Q is reduced coenzyme Q₁₀.

3. The antiaging composition according to claim 1,
which contains an oxidized coenzyme Q represented by the following formula (2): 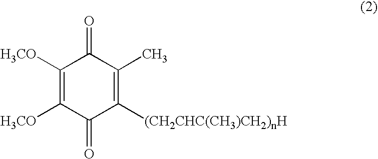
in the formula, n represents an integer of 1 to 12.

4. The antiaging composition according to claim 3,
wherein the oxidized coenzyme Q is oxidized coenzyme Q₁₀.

5. The antiaging composition according to claim 1,
wherein the content of the reduced coenzyme Q is 0.001 to 99% by weight.

6. A method of preventing an animal from aging
which comprises administering the antiaging composition according to claim 1 to the target animal.

7. A method of producing an antiaging composition
which comprises mixing a reduced coenzyme Q represented by the following formula (1): 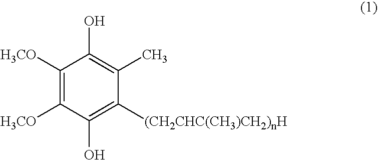
in the formula, n represents an integer of 1 to 12;
with an acceptable additive.

8. The antiaging composition according to claim 2, wherein the content of the reduced coenzyme Q is 0.001 to 99% by weight.

9. The antiaging composition according to claim 3, wherein the content of the reduced coenzyme Q is 0.001 to 99% by weight.

10. The antiaging composition according to claim 4, wherein the content of the reduced coenzyme Q is 0.001 to 99% by weight.

11. A method of preventing an animal from aging
which comprises administering the antiaging composition according to claim 2 to the target animal.

12. A method of preventing an animal from aging
which comprises administering the antiaging composition according to claim 3 to the target animal.

13. A method of preventing an animal from aging
which comprises administering the antiaging composition according to claim 4 to the target animal.

14. A method of preventing an animal from aging
which comprises administering the antiaging composition according to claim 5 to the target animal.
